(19) United States
(12) Patent Application Publication
(10) Pub. No.: US 2004/0147752 A1
(43) Pub. Date: Jul. 29, 2004

(54) Carbazole derivatives for the treatment of NPY related diseases

(75) Inventors: Klaus Rudolf, Warthausen (DE); Rudolf Hurnaus, Biberach (DE); Wolfgang Eberlein, Biberach (DE); Wolfhard Engel, Biberach (DE); Heike-Andrea Wieland, Biberach (DE); Bernd Krist, Ulm (DE)

Correspondence Address: BOEHRINGER INGELHEIM CORPORATION, 900 RIDGEBURY ROAD, P. O. BOX 368, RIDGEFIELD, CT, 06877, US

(73) Assignees: Boehringer Ingelheim International GmbH, Ingelheim (DE); Novo Nordisk A/S, Bagsvaerd (DK)

(21) Appl. No.: 10/753,776

(22) Filed: Jan. 8, 2004

Related U.S. Application Data
(63) Continuation of application No. 10/157,597, filed on May 29, 2002, now abandoned.
(60) Provisional application No. 60/307,675, filed on Jul. 25, 2001.

(30) Foreign Application Priority Data
May 29, 2001 (DE) 101 25 961

(51) Int. Cl. C 07D 4 3/02; C07D 209/82

(52) U.S. Cl. 546/199; 546/276.7; 548/444

(57) ABSTRACT

The invention relates to novel carbazole derivatives, their use for the preparation of a pharmaceutical composition for the treatment of eating and metabolic disorders such as obesity, bulimia nervosa, anorexia nervosa, of sleep disturbance, of morphine withdrawal symptoms and of epileptic seizures, a pharmaceutical composition containing them and a process for preparing them.

### RELATED APPLICATIONS

This application is a continuation of U.S. Nonprovisional application Ser. No. 10/157,597, files May 29, 2002, which claims benefit of U.S. Provisional Application Serial No. 60/307,675, filed on Jul. 25, 2001, and said applications are herein incorporated by reference in their entirety.

### FIELD OF THE INVENTION

The invention relates to novel carbazole derivatives and their use for the preparation of pharmaceutical compositions for the treatment of eating and metabolic disorders such as obesity, bulimia nervosa, anorexia nervosa, of sleep disturbance, of morphine withdrawal and of epileptic seizures.

### BACKGROUND OF THE INVENTION

Neuropeptide Y (NPY) is a 36 amino acid peptide discovered by Tatemoto in 1982 (Tatemoto, K. et al., Neuropeptide Y: complete amino acid sequence of the brain peptide. Proc. Natl. Acad. Sci. U.S.A. (1982), 79(18), 5485-9). Since its discovery, NPY has been found in the brain in concentrations higher than any other putative neurotransmitter. Hypothalamic regions are particularly rich in NPY-containing neurons, with the paraventricular nucleus containing perhaps the highest concentration of NPY in the brain.

Evidence suggests the existence of several NPY receptor subtypes among which hY1 (Larhammar et al., Cloning and functional expression of a human neuropeptide Y/peptide YY receptor of the Y1 type. J. Biol. Chem., 1992, 267(16), 10935-8.), hY2 (WO 95/21245), hY4 (WO 95/17906), hY5 (Gerald et al., Cloning and expression of a novel neuropeptide Y receptor. J. Biol. Chem. 271, 16435, 1996.; WO97/46250) and hY6 have been cloned (hY6: Weinberg et al., Cloning and expression of a novel neuropeptide Y receptor. J. Biol. Chem., 1996), 271(28), 16435-8). However, the Y6 receptor appears to be nonfunctional in humans. The NPY receptor subclassification is based mainly on the activity/affinity profile of NPY/PYY/PP and certain selective analogs/fragments (Michel M. C., et al., XVI. International Union of Pharmacology recommendations for the nomenclature of neuropeptide Y, peptide YY, and pancreatic polypeptide receptors. Pharmacol. Rev. March 1998; 50(1):143-50).

NPY is the most potent stimulant of food intake. Chronic i.c.v. administration of NPY in rats results in a robust increase in food intake associated with an increase in body weight and body fat content (White, Neuropeptide Y: a central regulator of energy homeostasis. Regul. Pept. 1993, 49(2), 93-107). Because NPY does not only increase food intake but also reduces energy expenditure it has been hypothesized that NPY could be an important brain peptide regulating energy balance. Moreover, food deprivation in rats is associated with an increase in NPY concentrations in the hypothalamus (Frankish et al., Neuropeptide Y, the hypothalamus, and diabetes: Insights into the central control of metabolism. Peptides, 16, 4, 757-771, 1995). This fluctuation of NPY levels in the brain with different feeding states supports a physiological role for NPY in feeding. Antisera against NPY (Dube M. G. et al., Evidence that neuropeptide Y is a physiological signal for normal food intake. Brain Res. (1994), 646(2), 341-4) as well as peptide (Myers et al, Anorexic action of a new potential neuropeptide Y antagonist [D-Tyr27,36,D-Thr32]-NPY (27-36) infused into the hypothalamus of the rat. Brain Res. Bull. 1995, 37(3), 237-45) and nonpeptide antagonists attenuate the hyperphagia seen after food deprivation. In addition, hypothalamic concentrations of NPY as well as NPY mRNA, are increased in genetically obese animals, such as the fatty Zucker rat or the ob/ob mouse (Frankish et al., Neuropeptide Y, the hypothalamus, and diabetes: Insights into the central control of metabolism. Peptides, 16, 4, 757 -771, 1995).

Accordingly, NPY antagonists seem to be promising candidates for the treatment of obesity. The characterization of the NPY receptor subtype responsible for food intake in rats is mainly based on functional experiments. The agonist receptor binding profile suggests that the Y5 receptor is involved in the NPY induced feeding behavior. Thus Y5 antagonists inhibit NPY mediated feeding as well as food intake in 24 hours food deprived rats (Criscione, L. et al., Food intake in free-feeding and energy-deprived lean rats is mediated by the neuropeptide Y5 receptor. J. Clin. Invest.1998, 102(12), 2136-2145; Kask et al. Neuropeptide Y Y5 receptor antagonist CGP71683A: the effects on food intake and anxiety-related behavior in the rat. Eur. J. Pharmacol., 2001, 414(2/3), 215-224) supporting the notion that the Y5 receptor is the “feeding” receptor. In addition, Hwa J. J. et al. (Activation of the NPY Y5 receptor regulates both feeding and energy expenditure. Am J Physiol 277(5 Pt 2), R1428-R1434, 1999) demonstrated a reduced oxygen consumption and energy expenditure in rats upon administration of a Y5 agonist, thereby further substantiating the role of the Y5 receptor in energy homeostasis. However, Y1 selective antagonists such as BIBO3304 and 1229U91 inhibit the NPY induced feeding in rodents and in primates (rhesus monkey experiments using 1229U91) too.

Other fields for the use of NPY Y5 receptor ligands have been demonstrated for the treatment of morphine withdrawal (Woldbye D. P. et al., Neuropeptide Y attenuates naloxone-precipitated morphine withdrawal via Y5-like receptors. J Pharmacol Exp Ther 284(2), 633-636, 1998), based on the attenuation of these effect upon i.c.v. administration of Y5 selective agonists, and for seizures, based on the reduced exhibition of spontaneous seizures in Y5 knock out mice (Marsh D. J. et al., Role of the Y5 neuropeptide Y receptor in limbic seizures. Proc Natl Acad Sci USA 96(23), 13518-13523, 1999) as well as administration of Y5 selective agonists (Woldbye D. P. et al., Powerful inhibition of kainic acid seizures by neuropeptide Y via Y5-like receptors. Nature Medicine, 1997, 3(7), 761-4) in seizure models. Influence of the Y5 receptors in the hypothalamic suprachiasmatic nucleus on the circadian rhythm have been reported by Gribkoff V. K. et al (Phase shifting of circadian rhythms and depression of neuronal activity in the rat suprachiasmatic nucleus by neuropeptide Y: mediation by different receptor subtypes. J. Neurosci. 18(8), 3014-3022, 1998).

### SUMMARY OF THE INVENTION

This invention is directed to novel carbazole compounds which bind selectively to and modulate, i.e. inhibit or stimulate, the activity of the human Y5 receptor. The invention relates to new compounds as listed in Table 1, or a salt thereof, to pharmaceutical compositions containing them, and to the manufacture of new compounds as listed in Table 1 and salts thereof. The invention furthermore relates to a method of treatment of disorders and diseases associated with NPY receptor subtype Y5, such as eating and metabolic disorders, of sleep disturbance, of morphine withdrawal and of epileptic seizures and to the use of the compounds according to the invention for the preparation of a pharmaceutical composition for treating said disorders and diseases.

### DETAILED DESCRIPTION OF THE INVENTION

It has now been found that the new compounds as listed in Table 1 and the diastereomers, enantiomers, mixtures and salts thereof, and in particular the physiologically acceptable salts thereof, are useful for the treatment of eating and metabolic disorders such as obesity, bulimia nervosa, anorexia nervosa, of sleep disturbance, of morphine withdrawal symptoms and of epileptic seizures.

TABLE 1
New compounds useful for the treatment of NPY related diseases

| No. | Chemical name | Chemical formula |
|---|---|---|
| 26-1 | N-(9-Ethyl-9H-carbazol-3-yl)-2-[4-(2- oxo-2,3-dihydro-benzoimidazol-1-yl)- piperidin-1-yl]-acetamide | 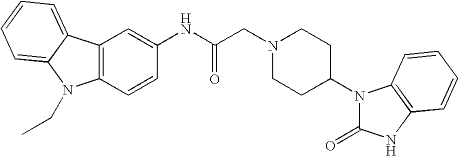 |
| 26-2 | N-(9-Ethyl-9H-carbazol-3-yl)-succinamic acid | 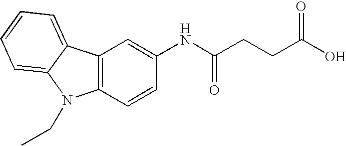 |
| 26-3 | Tetrahydro-furan-3-carboxylic acid (9- ethyl-9H-carbazol-3-yl)-amide | 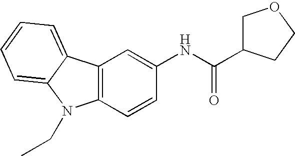 |
| 26-4 | N-(9-Ethyl-9H-carbazol-3-yl)-2-(2- methoxy-ethoxy)-acetamide | 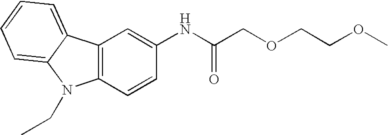 |
| 26-5 | N-(9-Ethyl-9H-carbazol-3-yl)-isonicotin- amide | 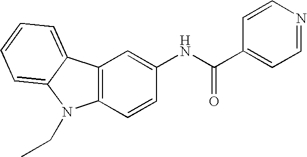 |
| 26-6 | N-(9-Ethyl-9H-carbazol-3-yl)- nicotinamide | 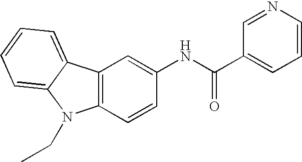 |
| 26-7 | Pyridine-2-carboxylic acid (9-ethyl-9H- carbazol-3-yl)-amide | 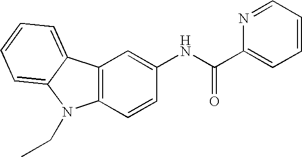 |
| 26-8 | N-(9-Ethyl-9H-carbazol-3-yl)-2-phenyl- acetamide | 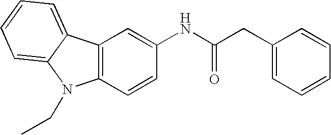 |
| 26-9 | N-(9-Ethyl-9H-carbazol-3-yl)-4-fluoro- benzamide | 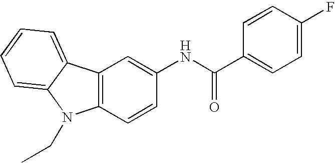 |
| 26-10 | 4-Chloro-N-(9-ethyl-9H-carbazol-3-yl)- benzamide | 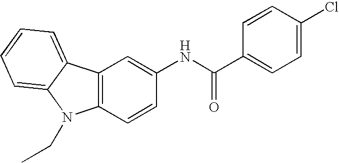 |
| 26-12 | 4-Dimethylamino-N-(9-ethyl-9H- carbazol-3-yl)-benzamide | 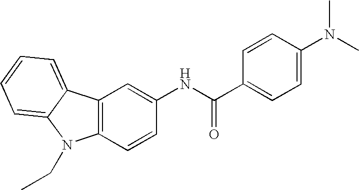 |
| 26-13 | N-(9-Ethyl-9H-carbazol-3-yl)-4-nitro- benzamide | 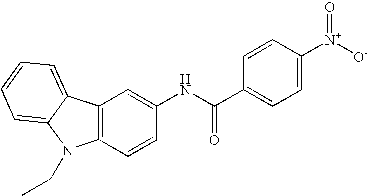 |
| 26-14 | 3-Chloro-N-(9-ethyl-9H-carbazol-3-yl)- benzamide | 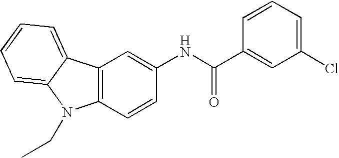 |
| 26-15 | (E)-N-(9-Ethyl-9H-carbazol-3-yl)-3- phenyl-acrylamide | 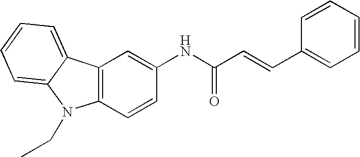 |
| 26-16 | N-(9-Ethyl-9H-carbazol-3-yl)-2,2- dimethyl-propionamide | 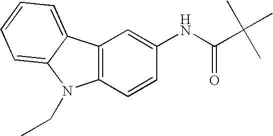 |
| 26-17 | N-(9-Ethyl-9H-carbazol-3-yl)-4-oxo-4- phenyl-butyramide | 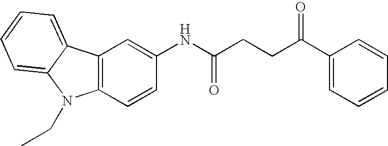 |
| 26-19 | 4-Methyl-[1,2,3]thiadiazole-5-carboxylic acid (9-ethyl-9H-carbazol-3-yl)-amide | 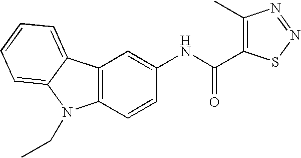 |
| 26-20 | 5-tert-Butyl-2-methyl-2H-pyrazole-3- carboxylic acid (9-ethyl-9H-carbazol-3- yl)-amide | 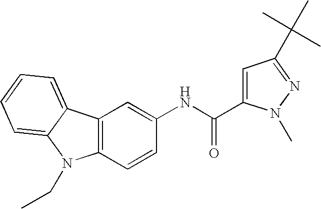 |
| 26-21 | 1-Methyl-1H-pyrrole-2-carboxylic acid (9-ethyl-9H-carbazol-3-yl)-amide | 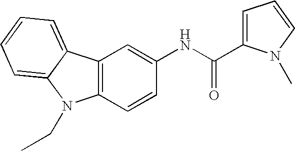 |
| 26-22 | Isoxazole-5-carboxylicacid (9-ethyl-9H- carbazol-3-yl)-amide | 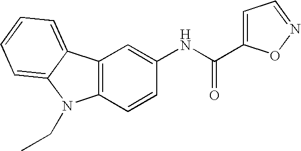 |
| 26-24 | 1H-Indole-2-carboxylic acid (9-ethyl-9H- carbazol-3-yl)-amide | 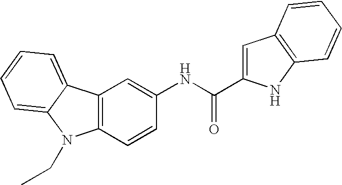 |
| 26-25 | N-(9-Ethyl-9H-carbazol-3-yl)-2-phenoxy- acetamide | 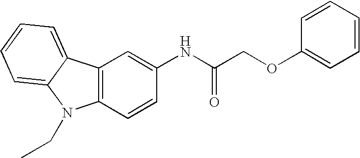 |
| 27-1 | 1-(9-Ethyl-9H-carbazol-3-yl)-3-isopropyl- urea | 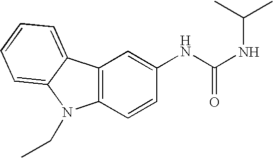 |
| 27-2 | 1-tert-Butyl-3-(9-ethyl-9H-carbazol-3-yl)- urea | 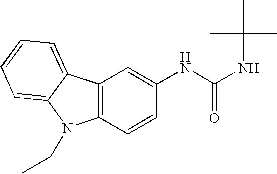 |
| 27-3 | 1-(9-Ethyl-9H-carbazol-3-yl)-3-(2- hydroxy-ethyl)-urea | 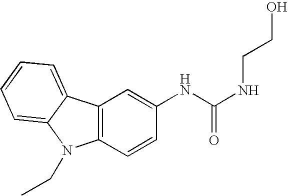 |
| 28-1 | 9-Ethyl-9H-carbazole-3-carboxylic acid phenylamide | 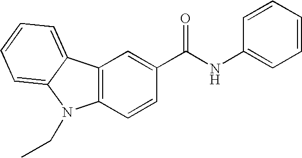 |
| 28-2 | 9-Ethyl-9H-carbazole-3-carboxylic acid isopropylamide | 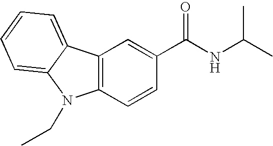 |
| 28-3 | 9-Ethyl-9H-carbazole-3-carboxylic acid tert-butylamide | 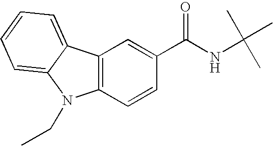 |
| 28-4 | 9-Ethyl-9H-carbazole-3-carboxylic acid diethylamide | 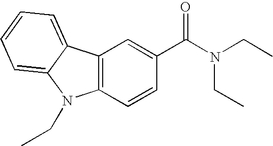 |
| 28-5 | 9-Ethyl-9H-carbazole-3-carboxylic acid cyclopropylamide | 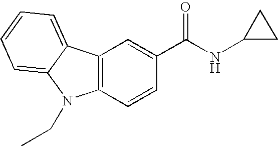 |
| 28-6 | 9-Ethyl-9H-carbazole-3-carboxylic acid (3-imidazol-1-yl-propyl)-amide | 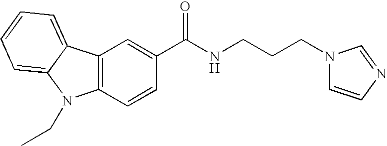 |
| 29-1 | N-(9-Methyl-9H-carbazol-3-yl)- benzamide | 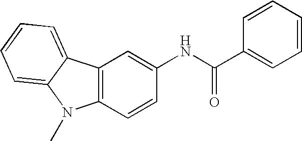 |
| 29-2 | 2-Dimethylamino-N-(9-methyl-9H- carbazol-3-yl)-acetamide | 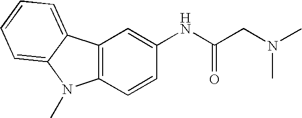 |
| 29-3 | 2-Methoxy-N-(9-methyl-9H-carbazol-3- yl)-acetamide | 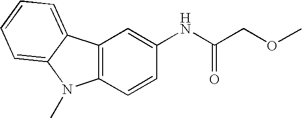 |
| 29-4 | N-(9-Methyl-9H-carbazol-3-yl)- succinamic acid | 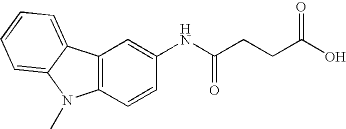 |
| 29-5 | N-(9-Methyl-9H-carbazol-3-yl)-2-phenyl- acetamide | 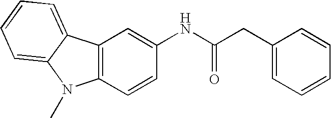 |
| 29-6 | N-(9-Methyl-9H-carbazol-3-yl)- \|isobutyramide\| | 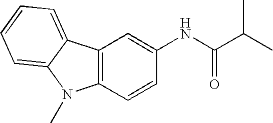 |
| 29-7 | 2,2-Dimethyl-N-(9-methyl-9H-carbazol- 3-yl)-propionamide | 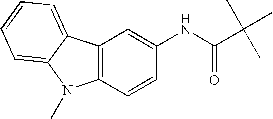 |
| 29-8 | Cyclohexanecarboxylic acid (9-methyl- 9H-carbazol-3-yl)-amide | 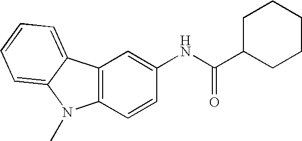 |
| 29-9 | Cyclopropanecarboxylic acid (9-methyl- 9H-carbazol-3-yl)-amide | 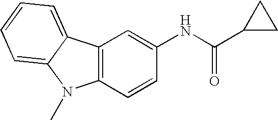 |
| 29-10 | N-(9-Methyl-9H-carbazol-3-yl)- isonicotinamide | 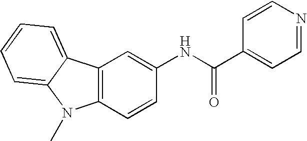 |
| 30-1 | N-(9-Benzyl-9H-carbazol-3-yl)- benzamide | 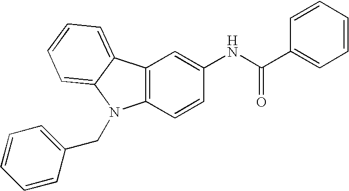 |
| 30-2 | N-(9-Benzyl-9H-carbazol-3-yl)-2- dimethylamino-acetamide | 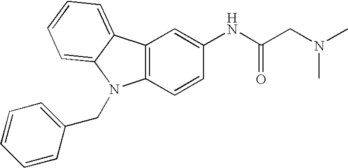 |
| 30-3 | N-(9-Benzyl-9H-carbazol-3-yl)-2- methoxy-acetamide | 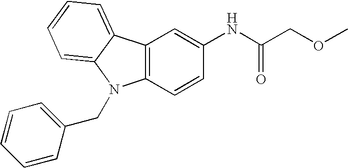 |
| 30-4 | N-(9-Benzyl-9H-carbazol-3-yl)- succinamic acid | 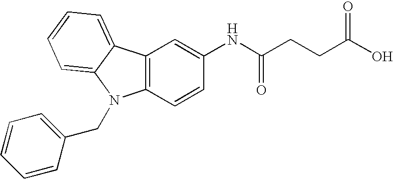 |
| 30-5 | N-(9-Benzyl-9H-carbazol-3-yl)-2-phenyl- acetamide | 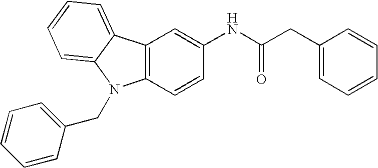 |
| 30-6 | N-(9-Benzyl-9H-carbazol-3-yl)-isobutyr- amide | 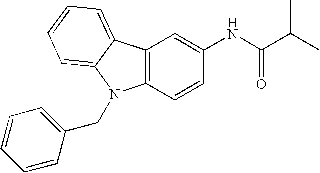 |
| 30-7 | N-(9-Benzyl-9H-carbazol-3-yl)-acetamide | 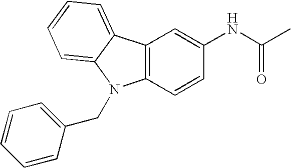 |
| 30-8 | Cyclohexanecarboxylic acid (9-benzyl- 9H-carbazol-3-yl)-amide | 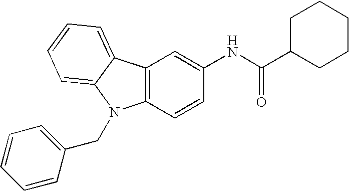 |
| 30-9 | Cyclopropanecarboxylic acid (9-benzyl- 9H-carbazol-3-yl)-amide | 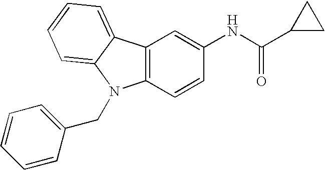 |
| 30-10 | N-(9-Benzyl-9H-carbazol-3-yl)- isonicotinamide | 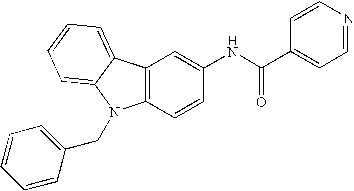 |
| 31-1 | N-(9H-Carbazol-3-yl)-2-dimethylamino- acetamide | 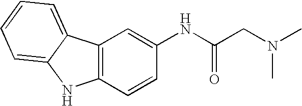 |
| 31-2 | N-(9H-Carbazol-3-yl)-2-methoxy- acetamide | 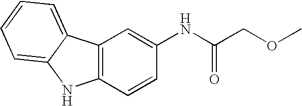 |
| 31-3 | N-(9H-Carbazol-3-yl)-succinamic acid | 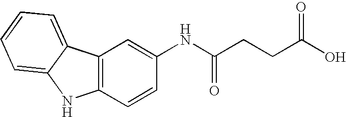 |
| 31-4 | N-(9H-Carbazol-3-yl)-2-phenyl- acetamide | 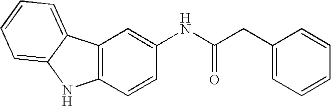 |
| 31-5 | N-(9H-Carbazol-3-yl)-isobutyramide | 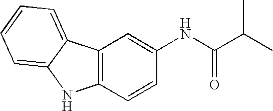 |
| 31-6 | Cyclohexanecarboxylic acid (9H- carbazol-3-yl)-amide | 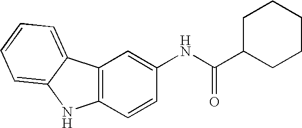 |
| 31-7 | Cyclopropanecarboxylic acid (9H- carbazol-3-yl)-amide | 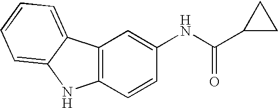 |
| 31-8 | N-(9H-Carbazol-3-yl)-isonicotinamide | 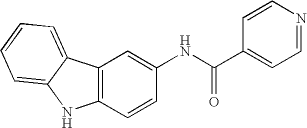 |

The present invention relates to the new compounds mentioned above, the diastereomers, enantiomers, mixtures and salts thereof, particularly to the pharmaceutically acceptable salts thereof, to their use for the treatment of eating and metabolic disorders such as obesity, bulimia nervosa, anorexia nervosa, of sleep disturbance, of morphine withdrawal symptoms and of epileptic seizures, to their use for the preparation of apharmaceutical composition for treating said disorders and diseases, pharmaceutical compositions containing them and processes for preparing them.

Preferred compounds are:

(a) tetrahydro-furan-3-carboxylic acid(9-ethyl-9H-carbazol-3-yl)-amide

(b) N-(9-ethyl-9H-carbazol-3-yl)-2-(2-methoxy-ethoxy)-acetamide

(c) N-(9-ethyl-9H-carbazol-3-yl)-nicotinamide

(d) N-(9-ethyl-9H-carbazol-3-yl)-2-phenyl-acetamide

(e) N-(9-ethyl-9H-carbazol-3-yl)-2,2-dimethyl-propionamide

(f) N-(9-ethyl-9H-carbazol-3-yl)-4-oxo4-phenyl-butyramide

(g) 2-chloro-N-(9-ethyl-9H-carbazol-3-yl)-benzamide

(h) 1-(9-ethyl-9H-carbazol-3-yl)-3-isopropyl-urea

(i) 1-(9-ethyl-9H-carbazol-3-yl)-3-(2-hydroxy-ethyl)-urea

(j) N-(9-methyl-9H-carbazol-3-yl)-isobutyramide

(k) 2,2-dimethyl-N-(9-methyl-9H-carbazol-3-yl)-propionamide

(l) cyclopropanecarboxylic acid(9-methyl-9H-carbazol-3-yl)-amide

and the diastereomers, enantiomers, mixtures and salts thereof, particularly to the pharmaceutically acceptable salts thereof.

The compounds according to the invention, if they contain a basic group, may be converted into their salts, particularly, for pharmaceutical use, into their physiologically acceptable salts, with inorganic or organic acids. Suitable acids for this purpose include, for example, hydrochloric acid, hydrobromic acid, sulfuric acid, phosphoric acid, fumaric acid, succinic acid, lactic acid, citric acid, tartaric acid or maleic acid.

Furthermore, the compounds according to the invention, if they contain a carboxy group, may, if desired, be converted subsequently into their salts with inorganic or organic bases, particularly, for pharmaceutical use, into their physiologically acceptable salts. Examples for suitable bases include sodium hydroxide, potassium hydroxide, cyclohexylamine, ethanolamine, diethanolamine and triethanolamine.

As already mentioned hereinbefore, the new compounds as listed in Table 1 and the salts thereof have valuable pharmacological properties and are useful for the treatment of eating and metabolic disorders such as obesity, bulimia nervosa, anorexia nervosa, of morphine withdrawal symptoms, of epileptic seizures or of sleep disturbance, particularly for the treatment of obesity.

In the Synthetic Section, the Following Abbreviations are Used:

Ac acetyl

aq. aqueous

DMSO dimethylsulfoxide

EDCI 1-ethyl-3-(3-dimethylaminopropyl)carbodiimid

Hünig's base ethyl-diisopropyl-amine

min. minutes

org. organic

PyCloP chlorotripyrrolidinophosphonium hexafluorophosphate

sat. saturated

CAN ceric ammonium nitrate

SiO₂ silica

Synthetic Methods

The syntheses of the key building blocks 18 and 22 to 24 are described in Scheme 1. Carbazol 25 was commercially available from Aldrich.

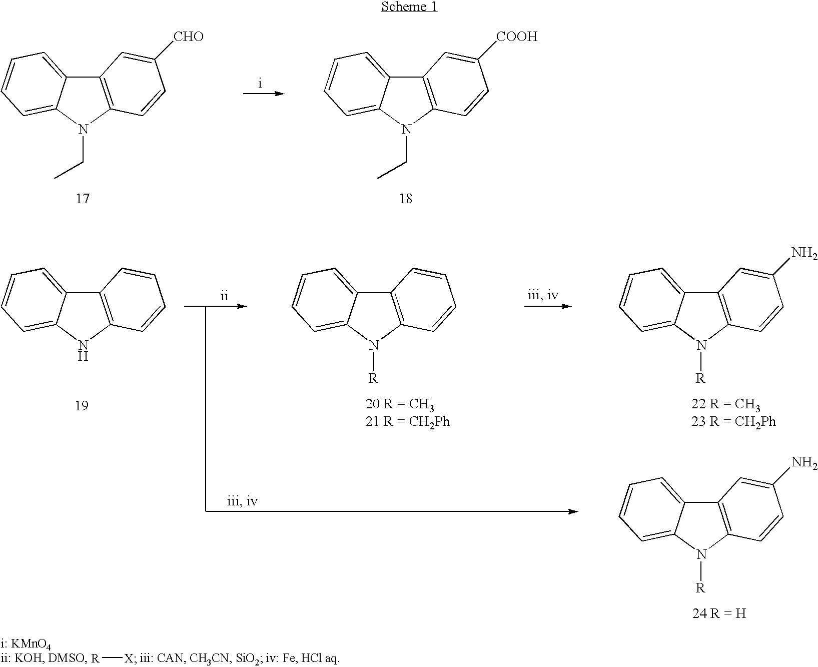

Procedure for the Synthesis of Acid 18:

To a stirred solution of 5.0 g Na₂CO₃ and 15.0 g KMnO₄ in water (250 ml) was added 5.0 g (22.4 mmol) 9-ethyl-3-carbazole carboxaldehyde. The reaction mixture was refluxed for 5 h, then allowed to cool to room temperature and treated with 10% aq. NaH₂PO₄— solution until pH 6 was reached. The reaction mixture was extracted with EtOAc, the org. layer washed with sat. Brine, dried (MgSO₄), evaporated and the residue dried under reduced pressure to yield 3.6 g of crude product, which after re-crystallization from EtOAc gave 1.49 g (27.8%) of acid 18.

1H-NMR (300 MHz, DMSO-d₆): 12.60 (br. S, 1H); 8.80 (m, 1H); 8.26 (dd, J=0.5, 7.2, 1H); 8.05 (dd, J=1.7, 8.7, 1H); 7.67-7.63 (m, 2H); 7.49 (dt, J=1.1, 7.1, 1H); 4.47 (q, J=7.1, 2H); 1.31 (t, J=7.1, 3H).

General Procedures for the Synthesis of Carbazoles 22 to 24:

Alkylation:

Was performed according to J Chem. Soc. Perkin Trans I, 1973, 499-500.

Nitration:

Was performed according to Synthetic Commun. 1994, 24, 1-10.

Reduction of the Nitro Group:

A mixture of 32% aq. HCI-solution (24 ml) and EtOH (24 ml) was added dropwise to a mixture of the corresponding 3-nitro-carbazole (11.3 mmol) and iron powder (6.31 g, 113 mmol) in EtOH (48 ml). The reaction mixture was stirred for 90 min. at 80° C., cooled and poured onto a mixture of 2N NaOH-solution (350 ml) and ice. The mixture was extracted with EtOAc, the org. layer washed with 2N aq. NaOH-solution and sat. Brine, dried (Na₂SO₄) and evaporated. The crude residue was suspended in Et₂O, filtered and dried under reduced pressure.

Yields: 22 (93.7%); 23 (83%); 24 (67.1%). 22: 1H-NMR (300MHz, DMSO-d₆): 9.91 (m, 1H); 7.44-7.25 (m, 4H); 7.05 (ddd, J=1.1, 6.9, 7.9, 1H); 6.81 (dd, J=2.0, 8.6, 1H); 4.71 (s, 2H); 3.74 (s, 3H). 23: 1H-NMR (300 MHz, DMSO-d₆): 7.95 (br. D, J=7.7, 1H); 7.47 (d, J=8.2, 1H), 7.33-7.04 (m, 9H); 6.77 (m (dd), 1H); 5.51 (s, 2H); 4.73 (s, 2H). 24: 1H-NMR (300 MHz, DMSO-d6): 10.67 (br. S, 1H); 7.87 (m, 1H); 7.35-7.14 (m, 4H); 7.00 (ddd, J=1.1, 6.8, 8.8, 1H); 6.74 (dd, J=2.2, 8.4, 1H); 4.65 (br. S, 2H).

General Procedure for the Preparation of 26 and 29, 30 and 31:

From Acid Chlorides:

To a solution of 3-amino carbazoles 22 to 25 (0.25 mmol) in CH₂Cl₂ (1 ml) was added pyridine (1.25 mmol) and N,N-dimethylaminopyridine (0.05 mmol) and the corresponding acid chloride (0.3 mmol). The reaction mixture was stirred for 2-24 h at room temperature, evaporated to dryness and the residue was chromatographed on SiO₂ using hexane/EtOAc.

From Carboxylic Acids:

To a solution of 3-amino carbazoles 22 to 25 (0.25 mmol) in CH₂Cl₂ (1 ml) was added the corresponding carboxylic acid (0.38 mmol) and EDCI (0.38 mmol) at room temperature. The reaction mixture was stirred for 24 h at room temperature and extracted with NaHCO₃-solution and EtOAc. The org. layer was dried (MgSO₄), evaporated and the residue chromatographed on SiO₂ as described above.

General Procedure for the Preparation of 27:

To 0.3 mmol of the corresponding amine or aniline in CH₃CN were added 0.11 mmol triphosgene and 0.9 mmol Hünig's base at 4° C. to give a clear solution, which was stirred at room temperature (30 min.) and for 2 h at 75° C. The reaction mixture was cooled to room temperature followed by addition of 25 (0.25 mmol). The reaction mixture was heated for 3 h at 75° C., evaporated to dryness and the residue was chromatographed on SiO₂ with hexane/EtOAc.

General Procedure for the Preparation of 28:

To a mixture of acid 18 (0.25 mmol) and the corresponding aniline or amine (0.28 mmol) in CH₂Cl₂ (1 ml) was added chlorotripyrrolidinophosphonium hexafluorophosphate (PyCloP) (0.28 mmol) and Hünig's base (1.05 mmol). The reaction mixture was stirred for 15 h at room temperature, extracted with water and EtOAc, the org. layer was dried (MgSO₄), evaporated and the residue chromatographed on SiO₂ with hexane/EtOAc.

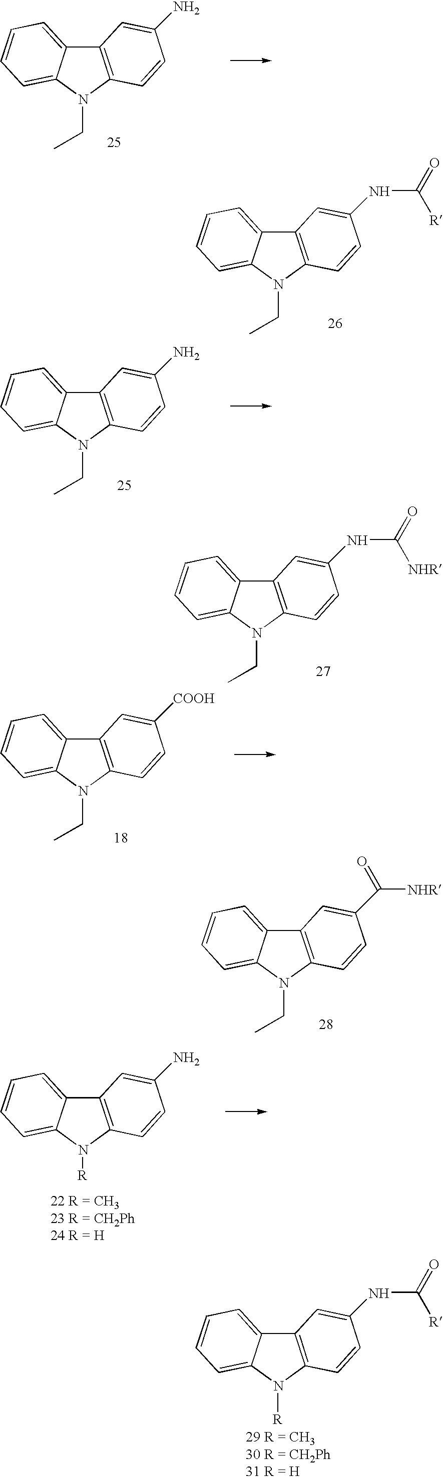

TABLE 2a
Examples for compounds of general formula 26:

| No. | Chemical name | Chemical formula |
|---|---|---|
| Example 26-10 | N-(9-Ethyl-9H-carbazol-3-yl)-2- [4-(2-oxo-2,3-dihydro- benzoimidazol-1-yl)-piperidin-1- yl]-acetamide | 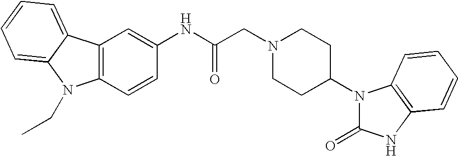 |
| Example 26-2 | N-(9-Ethyl-9H-carbazol-3-yl)- succinamic acid | 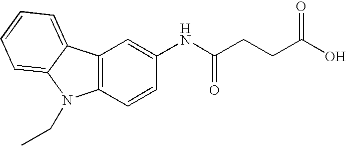 |
| Example 26-3 | Tetrahydro-furan-3-carboxylic acid (9-ethyl-9H-carbazol-3-yl)- amide | 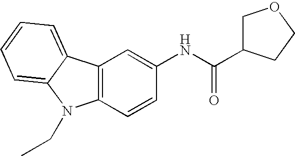 |
| Example 26-4 | N-(9-Ethyl-9H-carbazol-3-yl)-2- (2-methoxy-ethoxy)-acetamide | 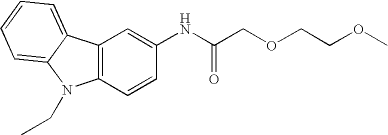 |
| Example 26-5 | N-(9-Ethyl-9H-carbazol-3-yl)- isonicotinamide | 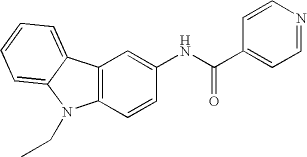 |
| Example 26-6 | N-(9-Ethyl-9H-carbazol-3-yl)- nicotinamide | 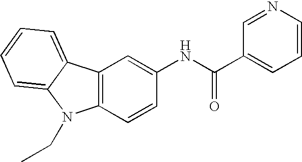 |
| Example 26-7 | Pyridine-2-carboxylic acid (9- ethyl-9H-carbazol-3-yl)-amide | 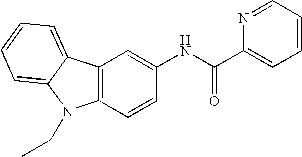 |
| Example 26-8 | N-(9-Ethyl-9H-carbazol-3-yl)-2- phenyl-acetamide | 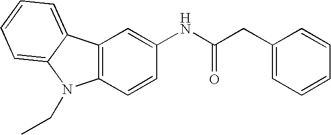 |
| Example 26-9 | N-(9-Ethyl-9H-carbazol-3-yl)-4- fluoro-benzamide | 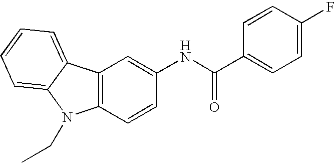 |
| Example 26-10 | 4-Chloro-N-(9-ethyl-9H- carbazol-3-yl)-benzamide | 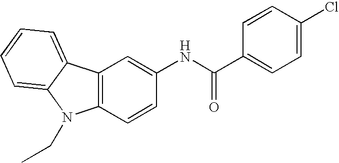 |
| Example 26-11 | N-(9-Ethyl-9H-carbazol-3-yl)-4- methoxy-benzamide | 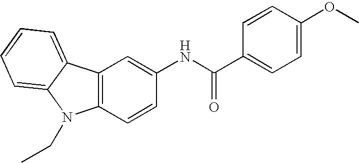 |
| Example 26-12 | 4-Dimethylamino-N-(9-ethyl- 9H-carbazol-3-yl)-benzamide | 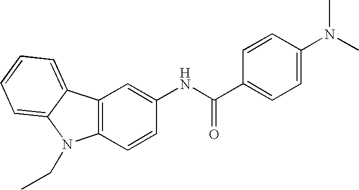 |
| Example 26-13 | N-(9-Ethyl-9H-carbazol-3-yl)-4- nitro-benzamide | 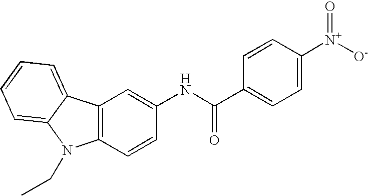 |
| Example 26-14 | 3-Chloro-N-(9-ethyl-9H- carbazol-3-yl)-benzamide | 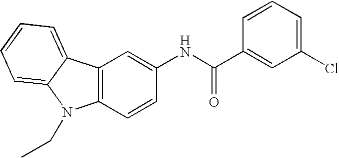 |
| Example 26-15 | (E)-N-(9-Ethyl-9H-carbazol-3- yl)-3-phenyl-acrylamide | 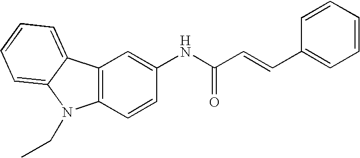 |
| Example 26-16 | N-(9-Ethyl-9H-carbazol-3-yl)- 2,2-dimethyl-propionamide | 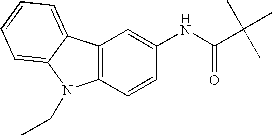 |
| Example 26-17 | N-(9-Ethyl-9H-carbazol-3-yl)-4- oxo-4-phenyl-butyramide | 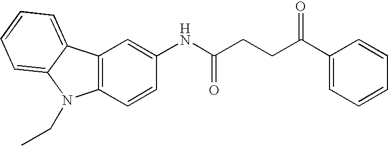 |
| Example 26-18 | 2-Chloro-N-(9-ethyl-9H- carbazol-3-yl)-benzamide | 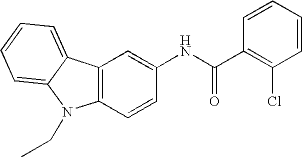 |
| Example 26-19 | 4-Methyl-[1,2,3]thiadiazole-5- carboxylic acid (9-ethyl-9H- carbazol-3-yl)-amide | 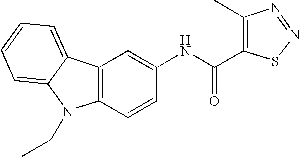 |
| Example 26-20 | 5-tert-Butyl-2-methyl-2H- pyrazole-3-carboxylic acid (9- ethyl-9H-carbazol-3-yl)-amide | 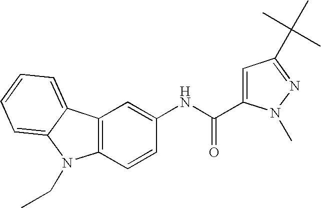 |
| Example 26-21 | 1-Methyl-1H-pyrrole-2- carboxylic acid (9-ethyl-9H- carbazol-3-yl)-amide | 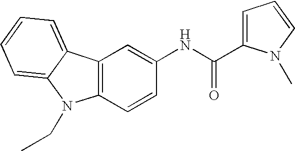 |
| Example 26-22 | Isoxazole-5-carboxylic acid (9- ethyl-9H-carbazol-3-yl)-amide | 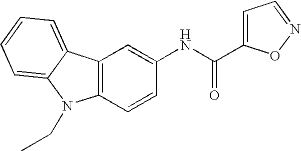 |
| Example 26-23 | Thiophene-2-carboxylic acid (9- ethyl-9H-carbazol-3-yl)-amide | 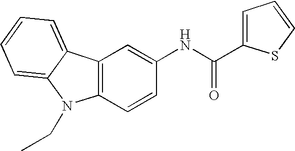 |
| Example 26-24 | 1H-Indole-2-carboxylic acid (9- ethyl-9H-carbazol-3-yl)-amide | 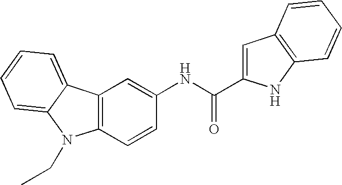 |
| Example 26-25 | N-(9-Ethyl-9H-carbazol-3-yl)-2- phenoxy-acetamide | 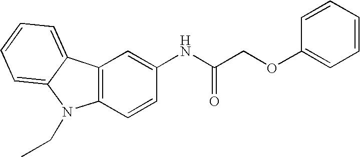 |

TABLE 2b
Experimental data of the compounds listed in table 2a

| | 1H NMR(300MHz) | | Mass Spectrum | | |
|---|---|---|---|---|---|
| | Solvent | signals | calculated | (M+H)⁺ | (M−H)⁺ |
| Example 26-1 | — | | 467,5756 | 468.5 | 466.5 |
| Example 26-2 | DMSO-d₆ | 1.28(t, 3H); 2.55(2t, 4H); | 310,3558 | 311.6 | 309.2 |
| | | 4.39(q, 2H); 7.12(t, 1H); 7.41 | | | |
| | | (t, 1H); 7.52(m, 3H); 8.01(d, | | | |
| | | 1H); 8.40(s, 1H); 9.98(s, | | | |
| | | 1H); 12.3(s, 1H) | | | |
| Example 26-3 | | | 308,3835 | 309.2 | 307.5 |
| Example 26-4 | DMSO-d₆ | 1.28(t, 3H); 3.30(s, 3H); 3.45 | 326,3988 | 327.4 | 325.6 |
| | | (q, 2H); 3.68(q, 2H); 4.09(s, | | | |
| | | 2H); 4.40(q, 2H); 7.16(t, | | | |
| | | 1H); 7.42(t, 1H); 7.68(m, | | | |
| | | 3H); 8.04(d, 1H); 8.40(d, | | | |
| | | 1H); 9.60(s, 1H) | | | |
| Example 26-5 | DMSO-d₆ | 1.29(t, 3H); 4.42(q, 2H); 7.20 | 315,378 | 316.4 | — |
| | | (t, 1H); 7.46(t, 1H); 7.62(t, | | | |
| | | 2H); 7.79(dd, 1H); 8.07(d, | | | |
| | | 1H); 8.30(m, 2H); 8.60(s, | | | |
| | | 1H); 8.97(m, 2H); 10.91(s, | | | |
| | | 1H) | | | |
| Example 26-6 | DMSO-d₆ | 1.31(t, 3H); 4.43(q, 2H); 7.13 | 315,378 | 316.3 | — |
| | | (t, 1H); 7.44(t, 1H); 7.58(m, | | | |
| | | 3H); 7.73(dd, 1H); 8.08(d, | | | |
| | | 1H); 8.30(dd, 1H); 8.52(s, | | | |
| | | 1H); 8.74(d, 1H); 9.16(s, | | | |
| | | 1H); 10.48(s, 1H) | | | |
| Example 26-7 | — | | 315,378 | 316.3 | — |
| Example 26-8 | — | | 328,4175 | 329.2 | — |
| Example 26-9 | DMSO-d₆ | 1.30(t, 3H); 4.43(q, 2H); 7.17 | 332,3808 | 333.4 | — |
| | | (t, 1H); 7.30-7.46(m, 3H); | | | |
| | | 7.58(d, 2H); 8.08(m, 3H); | | | |
| | | 8.31(s, 1H); 10.30(s, 1H) | | | |
| Example 26-10 | DMSO-d₆ | 1.28(t, 3H); 4.41(q, 2H); 7.17 | 348,8354 | 349.2 | — |
| | | (t, 1H); 7.23(t, 1H); 7.60(m, | | | |
| | | 3H); 7.73(d, 1H); 8.04(m, | | | |
| | | 3H); 8.51(s, 1H); 10.34(s, | | | |
| | | 1H) | | | |
| Example 26-11 | — | | 344,4169 | 345.2 | 343.4 |
| Example 26-12 | — | | 357,4593 | 358.4 | — |
| Example 26-13 | DMSO-d₆ | 1.30(t, 3H); 4.44(q, 2H); 7.18 | 359,3879 | 360.5 | — |
| | | (t, 1H); 7.44(t, 1H); 7.60(m, | | | |
| | | 2H); 7.75(dd, 1H); 8.08(d, | | | |
| | | 1H); 8.24(d, 2H); 8.37(d, | | | |
| | | 2H); 8.55(s, 1H); 10.61(s, | | | |
| | | 1H) | | | |
| Example 26-14 | — | | 348,8354 | 349.3 | 348.3 |
| Example 26-15 | — | | 340,4287 | 341.2 | — |
| Example 26-16 | DMSO-d₆ | 1.2-1.3(t + s; 12H); 4.39(q, | 294,4 | 295.2 | — |
| | | 2H); 7.15(t, 1H); 7.41(t, 1H); | | | |
| | | 7.5-7.6(m, 3H); 8.04(d, 1H); | | | |
| | | 8.32(d, 1H); 9.21(s, 1H) | | | |
| Example 26-17 | — | | 370,4551 | 371.5 | 369.5 |
| Example 26-18 | — | | 348,8354 | 349.2 | — |
| Example 26-19 | — | | 336,4184 | 337.6 | 335.7 |
| Example 26-20 | — | | 374,4899 | 375.2 | 373.2 |
| Example 26-21 | — | | 317,3939 | 318.2 | — |
| Example 26-22 | — | | 305,3392 | 306.4 | 304.2 |
| Example 26-23 | DMSO-d₆ | 1.30(t, 3H); 4.42(q, 2H); 7.20 | 320,4162 | 321.1 | — |
| | | (m, 2H); 7.45(t, 1H); 7.57(d, | | | |
| | | 2H); 7.71(dd, 1H); 7.83(d, | | | |
| | | 1H); 8.05(m, 2H); 1.45(s, | | | |
| | | 1H); 10.38(s, 1H) | | | |

| | | | | |
|---|---|---|---|---|
| Example 26-24 | — | 353,4274 | 354.3 | 352.5 |
| Example 26-25 | — | 344,4169 | 345.2 | — |

TABLE 3a
Examples for compounds of general formula 27

| No. | Chemical name | Chemical formula |
|---|---|---|
| Example 27-1 | 1-(9-Ethyl-9H-carbazol-3-yl)-3-isopropyl-urea | 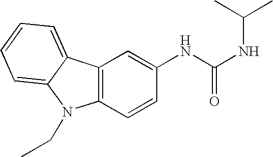 |
| Example 27-2 | 1-tert-Butyl-3-(9-ethyl-9H-carbazol-3-yl)-urea | 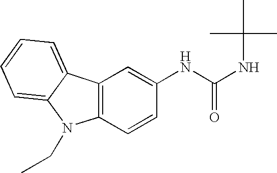 |
| Example 27-3 | 1-(9-Ethyl-9H-carbazol-3-yl)-3-(2-hydroxy-ethyl)-urea | 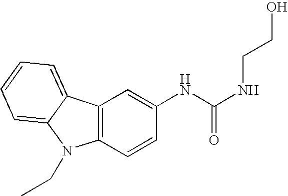 |

TABLE 3b
Experimental data of the compounds listed in table 3a

| | 1H NMR(300MHz) | | Mass Spectrum | | |
|---|---|---|---|---|---|
| | solvent | signals | calculated | (M+H)⁺ | (M−H)⁺ |
| Example | — | | 295,3876 | 296.1 | — |
| 27-1 | | | | | |
| Example | — | | 309,4147 | 310.6 | — |
| 27-2 | | | | | |
| Example | DMSO- | 1.26(t, 3H); 2.80(t, | 297,3599 | — | 297.6 |
| 27-3 | d₆ | 1H); 3.16(q, 2H); | | | |
| | | 3.45(m, 2H); | | | |
| | | 4.36(q, 2H); | | | |
| | | 4.73(m, 1H); | | | |
| | | 6.13(t, 1H); | | | |
| | | 7.12(t, 1H); | | | |
| | | 7.30-7.54 | | | |
| | | (m, 5H); 8.01(d, | | | |
| | | 1H); 8.15(d, 1H); | | | |
| | | 8.47(s, 1H) | | | |

TABLE 4a
Examples for compounds of general formula 28

| No. | Chemical name | Chemical formula |
|---|---|---|
| Example 28-1 | 9-Ethyl-9H-carbazole-3-carboxylic acid phenylamide | 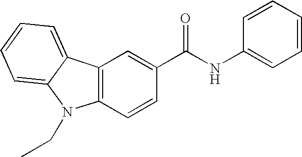 |
| Example 28-2 | 9-Ethyl-9H-carbazole-3-carboxylic acid isopropylamide | 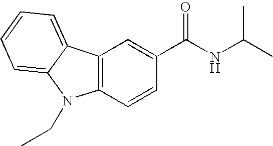 |
| Example 28-3 | 9-Ethyl-9H-carbazole-3-carboxylic acid tert-butylamide | 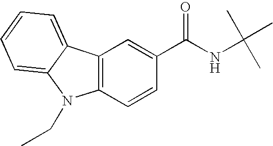 |
| Example 28-4 | 9-Ethyl-9H-carbazole-3-carboxylic acid diethylamide | 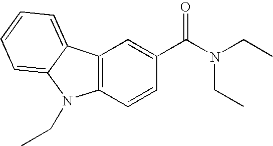 |
| Example 28-5 | 9-Ethyl-9H-carbazole-3-carboxylic acid cyclopropylamide | 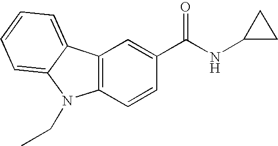 |
| Example 28-6 | 9-Ethyl-9H-carbazole-3-carboxylic acid (3-imidazol-1-yl-propyl)-amide | 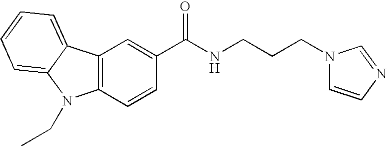 |

TABLE 4b
Experimental data of the compounds listed in table 4a

| | 1H NMR(300MHz) | | Mass Spectrum | | |
|---|---|---|---|---|---|
| | solvent | signals | calculated | (M+H)⁺ | (M−H)⁺ |
| Example 28-1 | — | | 314,3904 | 315.5 | 313.3 |
| Example 28-2 | — | | 280,3729 | 218.5 | — |
| Example 28-3 | — | | 294,4 | 295.5 | — |
| Example 28-4 | CDCl₃ | 1.23(t, 6H); 1.40(t, 3H); | 294,4 | 295.4 | — |
| | | 3.49(m, 4H); 4.37(q, | | | |
| | | 2H); 7.20-7.60(m, 4H); | | | |
| | | 8.08(d, 1H); 8.16(s, 1H) | | | |
| Example 28-5 | — | | 278,357 | 279.7 | — |
| Example 28-6 | CDCl₃ | 1.42(t, 3H); 2.20(p, 2H); | 346,436 | 347.4 | 345.1 |
| | | 3.55(q, 2H); 4.13(t, 2H); | | | |
| | | 4.35(q, 2H)6.71(t, 1H); | | | |
| | | 7.07(d, 2H); 7.20-7.55 | | | |
| | | (m, 4H); 7.90(m, 2H); 8.15(d, | | | |
| | | 1H); 8.55(s, 1H) | | | |

TABLE 5a
Examples for compounds of general formula 29

| No. | Chemical name | Chemical formula |
|---|---|---|
| Example 29-1 | N-(9-Methyl-9H-carbazol-3-yl)- benzamide | 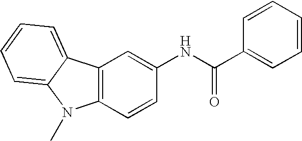 |
| Example 29-2 | 2-Dimethylamino-N-(9-methyl-9H- carbazol-3-yl)-acetamide | 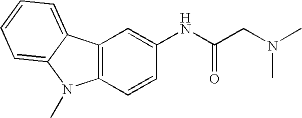 |
| Example 29-3 | 2-Methoxy-N-(9-methyl-9H-carbazol- 3-yl)-acetamide | 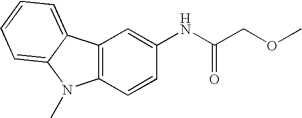 |
| Example 29-4 | N-(9-Methyl-9H-carbazol-3-yl)- succinamic acid | 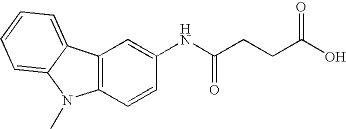 |
| Example 29-5 | N-(9-Methyl-9H-carbazol-3-yl)-2- phenyl-acetamide | 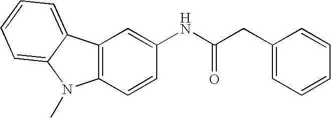 |
| Example 29-6 | N-(9-Methyl-9H-carbazol-3-yl)- isobutyramide | 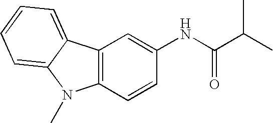 |
| Example 29-7 | 2,2-Dimethyl-N-(9-methyl-9H- carbazol-3-yl)-propionamide | 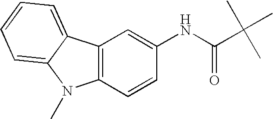 |
| Example 29-8 | Cyclohexanecarboxylic acid (9- methyl-9H-carbazol-3-yl)-amide | 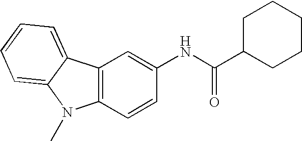 |
| Example 29-9 | Cyclopropanecarboxylic acid (9- methyl-9H-carbazol-3-yl)-amide | 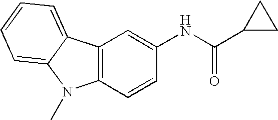 |
| Example 29-10 | N-(9-Methyl-9H-carbazol-3-yl)-iso- nicotinamide | 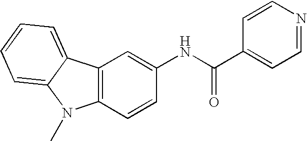 |

TABLE 5b
Experimental data of the compounds listed in table 5a

| | 1H NMR(300MHz) | | Mass Spectrum | | |
|---|---|---|---|---|---|
| | solvent | signals | calculated | (M+H)⁺ | (M−H)⁺ |
| Example | — | | 300,3633 | 301.6 | 299.2 |
| 29-1 | | | | | |
| Example | — | | 281,3605 | 282.3 | — |
| 29-2 | | | | | |
| Example | — | | 268,3181 | 269.6 | — |
| 29-3 | | | | | |
| Example | — | | 296,3287 | 297.4 | 295.3 |
| 29-4 | | | | | |
| Example | — | | 314,3904 | 315.1 | 312.6 |
| 29-5 | | | | | |
| Example | — | | 266,3458 | 267.6 | 366.6 |
| 29-6 | | | | | |
| Example | — | | 280,3729 | 281.5 | |
| 29-7 | | | | | |
| Example | — | | 306,4111 | 307.4 | 305.4 |
| 29-8 | | | | | |
| Example | CDCl₃ | 0.78(m, 2H); | 264,3299 | 265.5 | — |
| 29-9 | | 1.06(q, 2H); | | | |
| | | 1.47(q, 1H); 3.73(s, | | | |
| | | 3H); 7.07-7.48(m, | | | |
| | | 5H); 7.96(d, 1H); | | | |
| | | 8.26(s, 1H) | | | |

| | | | | |
|---|---|---|---|---|
| Example | — | 301,3509 | 302.6 | 300.0 |
| 29-10 | | | | |

TABLE 6a
Examples for compounds of general formula 30

| No. | Chemical name | Chemical formula |
|---|---|---|
| Example 30-1 | N-(9-Benzyl-9H-carbazol-3-yl)- benzamide | 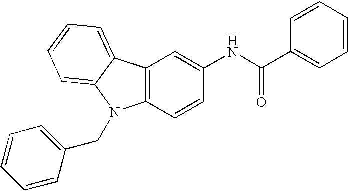 |
| Example 30-2 | N-(9-Benzyl-9H-carbazol-3-yl)-2- dimethylamino-acetamide | 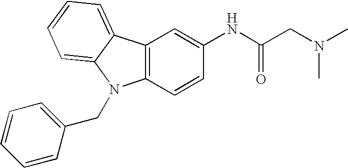 |
| Example 30-3 | N-(9-Benzyl-9H-carbazol-3-yl)-2- methoxy-acetamide | 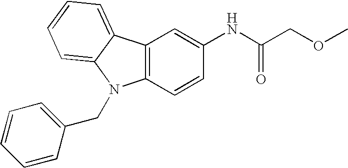 |
| Example 30-4 | N-(9-Benzyl-9H-carbazol-3-yl)- succinamic acid | 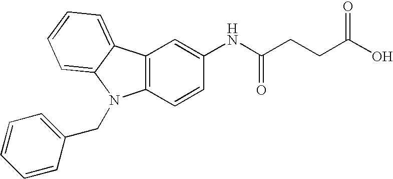 |
| Example 30-5 | N-(9-Benzyl-9H-carbazol-3-yl)-2- phenyl-acetamide | 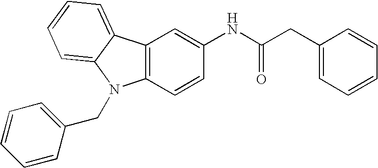 |
| Example 30-6 | N-(9-Benzyl-9H-carbazol-3-yl)- isobutyramide | 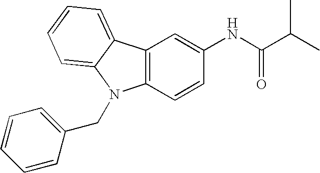 |
| Example 30-7 | N-(9-Benzyl-9H-carbazol-3-yl)- acetamide | 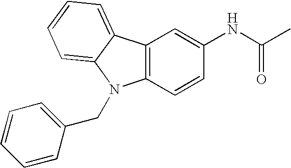 |
| Example 30-8 | Cyclohexanecarboxylic acid (9- benzyl-9H-carbazol-3-yl)-amide | 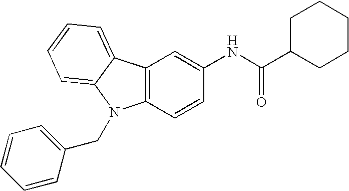 |
| Example 30-9 | Cyclopropanecarboxylic acid (9- benzyl-9H-carbazol-3-yl)-amide | 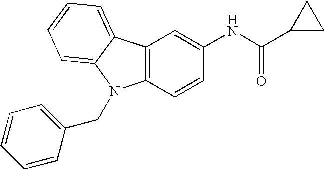 |
| Example 30-10 | N-(9-Benzyl-9H-carbazol-3-yl)- isonicotinamide | 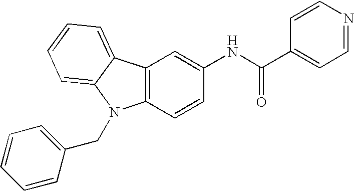 |

TABLE 6b
Experimental data of the compounds listed in table 6a

| | 1H NMR(300MHz) | | Mass Spectrum | | |
|---|---|---|---|---|---|
| | solvent | signals | calculated | (M+H)⁺ | (M−H)⁺ |
| Example 30-1 | — | | 376,4621 | 377.4 | 375.2 |
| Example 30-2 | — | | 357,4593 | 358.3 | — |
| Example 30-3 | CDCl₃ | 3.53(s, 3H); 4.09(s, 2H); | 344,4169 | 345.3 | 343.4 |
| | | 5.51(s, 2H); 7.08-7.55(m, 10H); | | | |
| | | 8.11(d, 1H); 8.40(m, 2H) | | | |

| | | | | |
|---|---|---|---|---|
| Example 30-4 | — | 372,4275 | 373.0 | 371.2 |
| Example 30-5 | — | 390,4892 | 391.8 | 388.8 |
| Example 30-6 | — | 342,4446 | 343.3 | — |
| Example 30-7 | — | 314,3904 | 315.0 | — |
| Example 30-8 | — | 382,5099 | 383.3 | 382.0 |
| Example 30-9 | — | 340,4287 | 341.2 | — |
| Example 30-10 | — | 377,4497 | 378.3 | 376.1 |

TABLE 7a
Examples for compounds of general formula 31

| No. | Chemical name | Chemical formula |
|---|---|---|
| Example 31-1 | N-(9H-Carbazol-3-yl)-2-dimethyl- amino-acetamide | 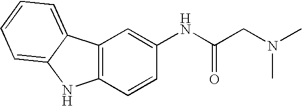 |
| Example 31-2 | N-(9H-Carbazol-3-yl)-2-methoxy- acetamide | 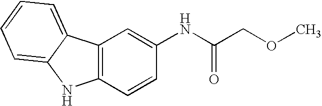 |
| Example 31-3 | N-(9H-Carbazol-3-yl)-succinamic acid | 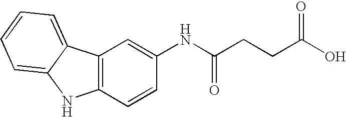 |
| Example 31-4 | N-(9H-Carbazol-3-yl)-2-phenyl- acetamide | 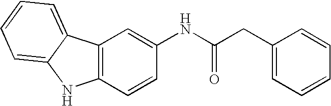 |
| Example 31-5 | N-(9H-Carbazol-3-yl)-isobutyramide | 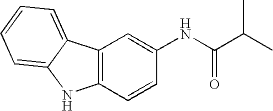 |
| Example 31-6 | Cyclohexanecarboxylic acid (9H- carbazol-3-yl)-amide | 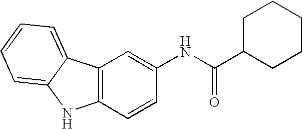 |
| Example 31-7 | Cyclopropanecarboxylic acid (9H- carbazol-3-yl)-amide | 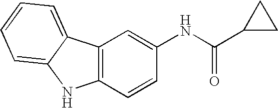 |
| Example 31-8 | N-(9H-Carbazol-3-yl)-isonicotin- amide | 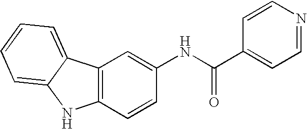 |

TABLE 7b
Experimental data of the compounds listed in table 7a

| | 1H NMR(300MHz) | | Mass Spectrum | | |
|---|---|---|---|---|---|
| | solvent | signals | calculated | (M+H)⁺ | (M−H)⁺ |
| Example 31-1 | DMSO-d₆ | 2.28(s, 6H); 3.08(s, | 267,3334 | 268.5 | 266.3 |
| | | 2H); 7.10(t, 1H); 7.30-7.46 | | | |
| | | (m, 3H); 7.56(dd, | | | |
| | | 1H); 8.00(d, 1H); 9.65 | | | |
| | | (s, 1H); 11.14(s, 1H) | | | |
| Example 31-2 | DMSO-d₆ | 3.40(s, 3H); 4.00(s, | 254,291 | 255.4 | 253.3 |
| | | 2H); 7.11(t, 1H); 7.30-7.50 | | | |
| | | (m, 3H); 7.56(dd, | | | |
| | | 1H); 8.00(d, 1H); 8.37 | | | |
| | | (s, 1H); 9.69(s, 1H); | | | |
| | | 11.16(s, 1H) | | | |

| | | | | |
|---|---|---|---|---|
| Example 31-3 | — | 282,3016 | 283.6 | 281.2 |
| Example 31-4 | — | 300,3633 | 301.4 | 299.2 |
| Example 31-5 | — | 252,3187 | 253.4 | 251.0 |
| Example 31-6 | — | 292,3841 | 293.6 | 291.1 |
| Example 31-7 | — | 250,3028 | 251.3 | 249.2 |
| Example 31-8 | — | 287,3238 | 288.3 | 285.9 |

We claim:

1. A compound selected from Table 1:
TABLE 1

| No. | Chemical name | Chemical formula |
|---|---|---|
| 26-1 | N-(9-Ethyl-9H-carbazol-3-yl)-2-[4-(2- oxo-2,3-dihydro-benzoimidazol-1-yl)- piperidin-1-yl]-acetamide | 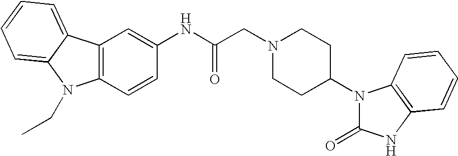 |
| 26-2 | N-(9-Ethyl-9H-carbazol-3-yl)-succinamic acid | 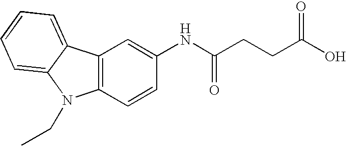 |
| 26-3 | Tetrahydro-furan-3-carboxylic acid (9- ethyl-9H-carbazol-3-yl)-amide | 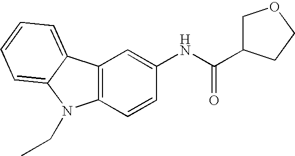 |
| 26-4 | N-(9-Ethyl-9H-carbazol-3-yl)-2-(2- methoxy-ethoxy)-acetamide | 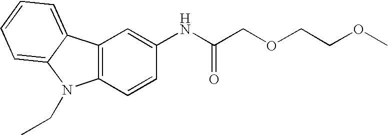 |
| 26-5 | N-(9-Ethyl-9H-carbazol-3-yl)-isonicotin- amide | 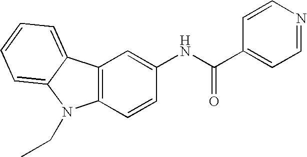 |
| 26-6 | N-(9-Ethyl-9H-carbazol-3-yl)- nicotinamide | 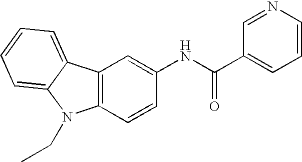 |
| 26-7 | Pyridine-2-carboxylic acid (9-ethyl-9H- carbazol-3-yl)-amide | 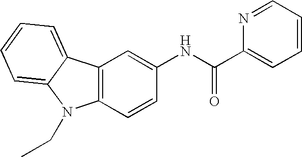 |
| 26-8 | N-(9-Ethyl-9H-carbazol-3-yl)-2-phenyl- acetamide | 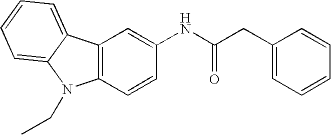 |
| 26-9 | N-(9-Ethyl-9H-carbazol-3-yl)-4-fluoro- benzamide | 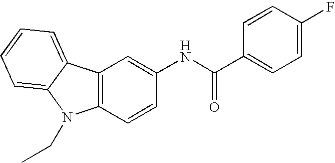 |
| 26-10 | 4-Chloro-N-(9-ethyl-9H-carbazol-3-yl)- benzamide | 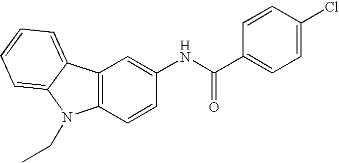 |
| 26-12 | 4-Dimethylamino-N-(9-ethyl-9H- carbazol-3-yl)-benzamide | 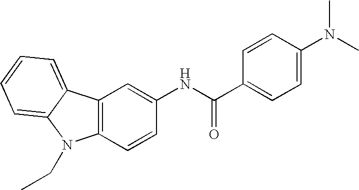 |
| 26-13 | N-(9-Ethyl-9H-carbazol-3-yl)-4-nitro- benzamide | 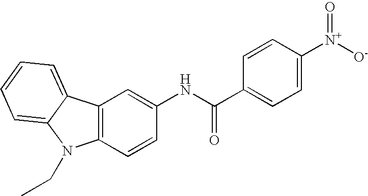 |
| 26-14 | 3-Chloro-N-(9-ethyl-9H-carbazol-3-yl)- benzamide | 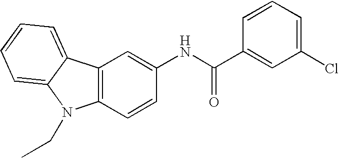 |
| 26-15 | (E)-N-(9-Ethyl-9H-carbazol-3-yl)-3- phenyl-acrylamide | 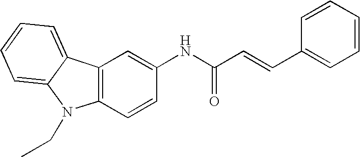 |
| 26-16 | N-(9-Ethyl-9H-carbazol-3-yl)-2,2- dimethyl-propionamide | 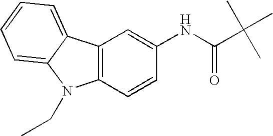 |
| 26-17 | N-(9-Ethyl-9H-carbazol-3-yl)-4-oxo-4- phenyl-butyramide | 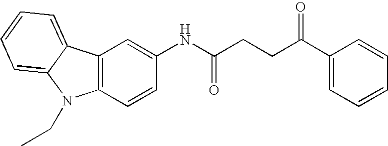 |
| 26-19 | 4-Methyl-[1,2,3]thiadiazole-5-carboxylic acid (9-ethyl-9H-carbazol-3-yl)-amide | 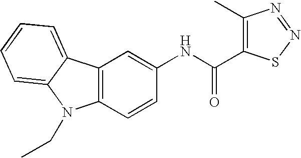 |
| 26-20 | 5-tert-Butyl-2-methyl-2H-pyrazole-3- carboxylic acid (9-ethyl-9H-carbazol-3- yl)-amide | 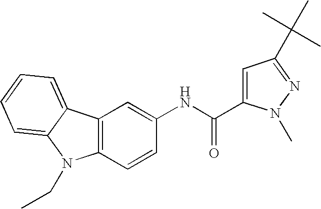 |
| 26-21 | 1-Methyl-1H-pyrrole-2-carboxylic acid (9-ethyl-9H-carbazol-3-yl)-amide | 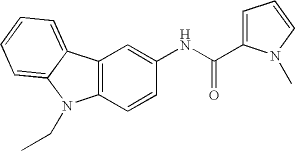 |
| 26-22 | Isoxazole-5-carboxylicacid (9-ethyl-9H- carbazol-3-yl)-amide | 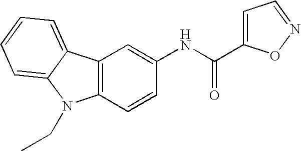 |
| 26-24 | 1H-Indole-2-carboxylic acid (9-ethyl-9H- carbazol-3-yl)-amide | 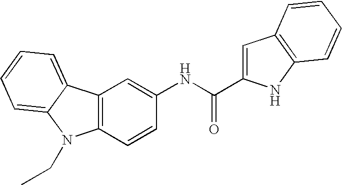 |
| 26-25 | N-(9-Ethyl-9H-carbazol-3-yl)-2-phenoxy- acetamide | 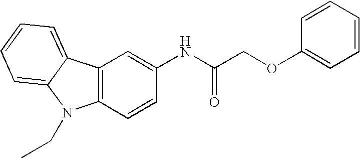 |
| 27-1 | 1-(9-Ethyl-9H-carbazol-3-yl)-3-isopropyl- urea | 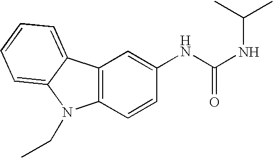 |
| 27-2 | 1-tert-Butyl-3-(9-ethyl-9H-carbazol-3-yl)- urea | 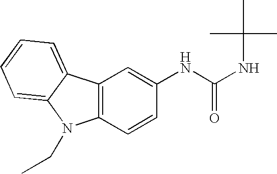 |
| 27-3 | 1-(9-Ethyl-9H-carbazol-3-yl)-3-(2- hydroxy-ethyl)-urea | 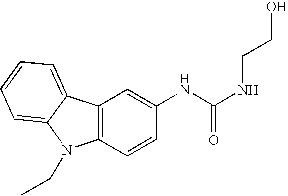 |
| 28-1 | 9-Ethyl-9H-carbazole-3-carboxylic acid phenylamide | 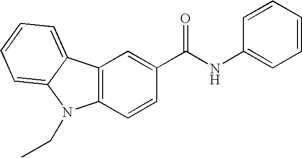 |
| 28-2 | 9-Ethyl-9H-carbazole-3-carboxylic acid isopropylamide | 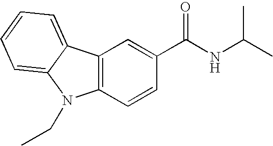 |
| 28-3 | 9-Ethyl-9H-carbazole-3-carboxylic acid tert-butylamide | 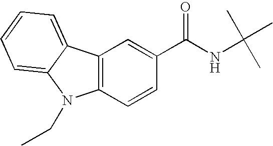 |
| 28-4 | 9-Ethyl-9H-carbazole-3-carboxylic acid diethylamide | 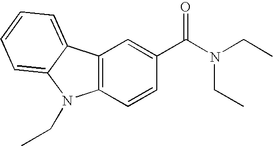 |
| 28-5 | 9-Ethyl-9H-carbazole-3-carboxylic acid cyclopropylamide | 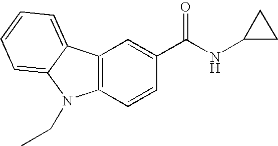 |
| 28-6 | 9-Ethyl-9H-carbazole-3-carboxylic acid (3-imidazol-1-yl-propyl)-amide | 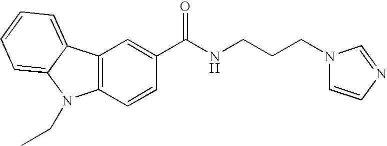 |
| 29-1 | N-(9-Methyl-9H-carbazol-3-yl)- benzamide | 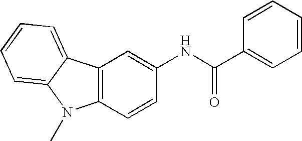 |
| 29-2 | 2-Dimethylamino-N-(9-methyl-9H- carbazol-3-yl)-acetamide | 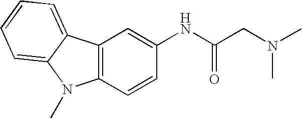 |
| 29-3 | 2-Methoxy-N-(9-methyl-9H-carbazol-3- yl)-acetamide | 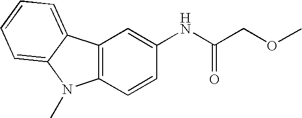 |
| 29-4 | N-(9-Methyl-9H-carbazol-3-yl)- succinamic acid | 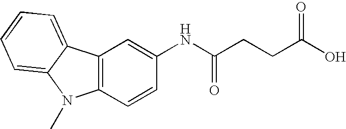 |
| 29-5 | N-(9-Methyl-9H-carbazol-3-yl)-2-phenyl- acetamide | 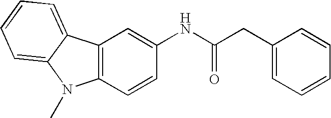 |
| 29-6 | N-(9-Methyl-9H-carbazol-3-yl)- \|isobutyramide\| | 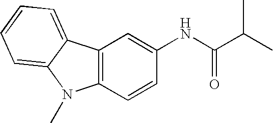 |
| 29-7 | 2,2-Dimethyl-N-(9-methyl-9H-carbazol- 3-yl)-propionamide | 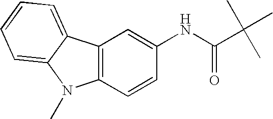 |
| 29-8 | Cyclohexanecarboxylic acid (9-methyl- 9H-carbazol-3-yl)-amide | 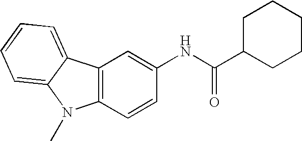 |
| 29-9 | Cyclopropanecarboxylic acid (9-methyl- 9H-carbazol-3-yl)-amide | 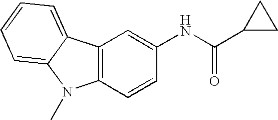 |
| 29-10 | N-(9-Methyl-9H-carbazol-3-yl)- isonicotinamide | 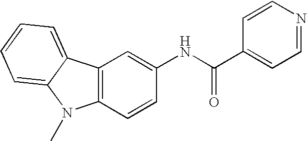 |
| 30-1 | N-(9-Benzyl-9H-carbazol-3-yl)- benzamide | 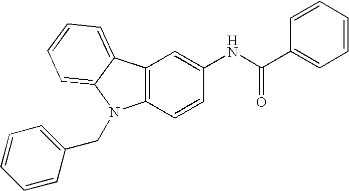 |
| 30-2 | N-(9-Benzyl-9H-carbazol-3-yl)-2- dimethylamino-acetamide | 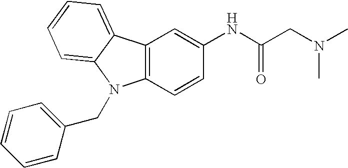 |
| 30-3 | N-(9-Benzyl-9H-carbazol-3-yl)-2- methoxy-acetamide | 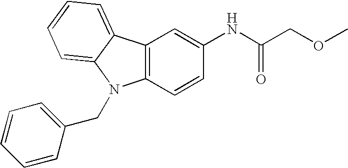 |
| 30-4 | N-(9-Benzyl-9H-carbazol-3-yl)- succinamic acid | 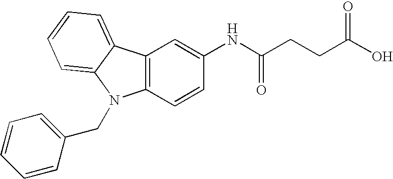 |
| 30-5 | N-(9-Benzyl-9H-carbazol-3-yl)-2-phenyl- acetamide | 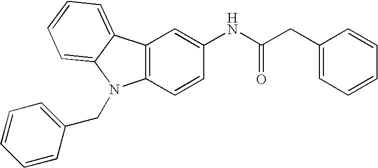 |
| 30-6 | N-(9-Benzyl-9H-carbazol-3-yl)-isobutyr- amide | 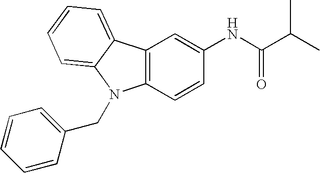 |
| 30-7 | N-(9-Benzyl-9H-carbazol-3-yl)-acetamide | 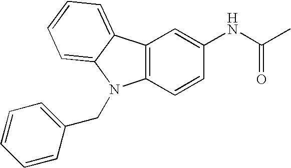 |
| 30-8 | Cyclohexanecarboxylic acid (9-benzyl- 9H-carbazol-3-yl)-amide | 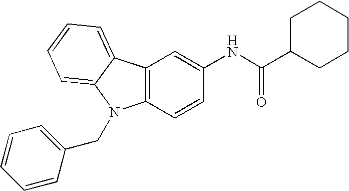 |
| 30-9 | Cyclopropanecarboxylic acid (9-benzyl- 9H-carbazol-3-yl)-amide | 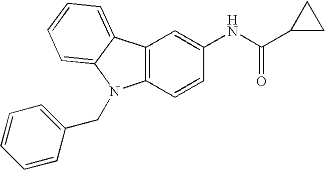 |
| 30-10 | N-(9-Benzyl-9H-carbazol-3-yl)- isonicotinamide | 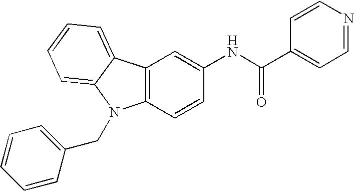 |
| 31-1 | N-(9H-Carbazol-3-yl)-2-dimethylamino- acetamide | 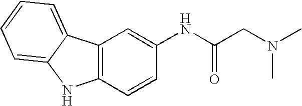 |
| 31-2 | N-(9H-Carbazol-3-yl)-2-methoxy- acetamide | 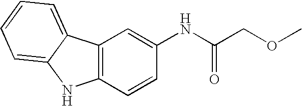 |
| 31-3 | N-(9H-Carbazol-3-yl)-succinamic acid | 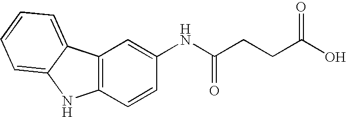 |
| 31-4 | N-(9H-Carbazol-3-yl)-2-phenyl- acetamide | 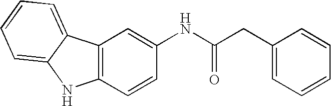 |
| 31-5 | N-(9H-Carbazol-3-yl)-isobutyramide | 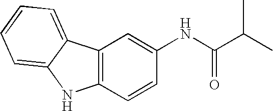 |
| 31-6 | Cyclohexanecarboxylic acid (9H- carbazol-3-yl)-amide | 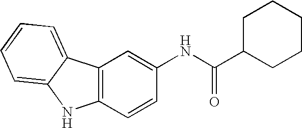 |
| 31-7 | Cyclopropanecarboxylic acid (9H- carbazol-3-yl)-amide | 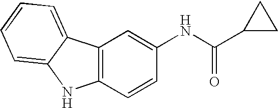 |
| 31-8 | N-(9H-Carbazol-3-yl)-isonicotinamide | 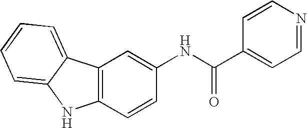 |

or a diastereomer, enantiomer, mixture or salt thereof.

2. A compound according to claim 1 selected from the following compounds:
(a) tetrahydro-furan-3-carboxylic acid(9-ethyl-9H-carbazol-3-yl)-amide;
(b) N-(9-ethyl-9H-carbazol-3-yl)-2-(2-methoxy-ethoxy)-acetamide;
(c) N-(9-ethyl-9H-carbazol-3-yl)-nicotinamide;
(d) N-(9-ethyl-9H-carbazol-3-yl)-2-phenyl-acetamide;
(e) N-(9-ethyl-9H-carbazol-3-yl)-2,2-dimethyl-propionamide;
(f) N-(9-ethyl-9H-carbazol-3-yl)-4-oxo-4-phenyl-butyramide;
(g) 2-chloro-N-(9-ethyl-9H-carbazol-3-yl)-benzamide;
(h) 1-(9-ethyl-9H-carbazol-3-yl)-3-isopropyl-urea;
(i) 1-(9-ethyl-9H-carbazol-3-yl)-3-(2-hydroxy-ethyl)-urea;
(j) N-(9-methyl-9H-carbazol-3-yl)-isobutyramide;
(k) 2,2-dimethyl-N-(9-methyl-9H-carbazol-3-yl)-propionamide; and
(l) cyclopropanecarboxylic acid(9-methyl-9H-carbazol-3-yl)-amide;
or a diastereomer, enantiomer, mixture or salt thereof.

3. A compound according to claim 1 in the form of a pharmaceutically acceptable salt.

4. A compound according to claim 2 in the form of a pharmaceutically acceptable salt.

5. A pharmaceutical composition comprising a compound according to claim 1 or a pharmaceutically acceptable salt thereof.

6. A pharmaceutical composition comprising a compound according to claim 2 or a pharmaceutically acceptable salt thereof.

7. A method for treating an eating or metabolic disorder which comprises administering to a patient in need of such treatment a therapeutically effective amount of a compound according to claim 1 or a pharmaceutically acceptable salt thereof.

8. A method for treating an eating or metabolic disorder which comprises administering to a patient in need of such treatment a therapeutically effective amount of a compound according to claim 2 or a pharmaceutically acceptable salt thereof.

9. A method according to claim 7, wherein the disorder is selected from obesity, bulimia nervosa and anorexia nervosa.

10. A method according to claim 8, wherein the disorder is selected from obesity, bulimia nervosa and anorexia nervosa.

11. A method for treating sleep disturbance, morphine withdrawal symptoms or epileptic seizures which comprises administering to a patient in need of such treatment a therapeutically effective amount of a compound according to claim 1 or a pharmaceutically acceptable salt thereof.

12. A method for treating sleep disturbance, morphine withdrawal symptoms or epileptic seizures which comprises administering to a patient in need of such treatment a therapeutically effective amount of a compound according to claim 2 or a pharmaceutically acceptable salt thereof.
